(19) United States
(12) Patent Application Publication
(10) Pub. No.: US 2009/0005296 A1
(43) Pub. Date: Jan. 1, 2009

(54) Ammonium/Ammonia Transporter

(75) Inventors: Thomas Jahn, Kobenhavn (DK); Jan Kofod Schjoerring, Bagsvaerd (DK); Dan Klaerke, Copenhagen (DK); Thomas Zeuthen, Virum (DK)

Correspondence Address: MICHAEL BEST & FRIEDRICH LLP, ONE SOUTH PINCKNEY STREET, P O BOX 1806, MADISON, WI, 53701, US

(73) Assignee: THE ROYAL VETERINARY AND AGRICULTURAL UNIVERSITY, Copenhagen (DK)

(21) Appl. No.: 11/568,691

(22) PCT Filed: May 4, 2005
(86) PCT No.: PCT/IB2005/001709

§ 371 Date: Feb. 7, 2007

Related U.S. Application Data
(60) Provisional application No. 60/567,792, filed on May 5, 2004.

(51) Int. Cl. A61K 38/16; C07K 14/46; A01H 5/00; C12Q 1/02; C12Q 1/68; A61P 3/00

(52) U.S. Cl. 514/12; 530/402; 800/298; 435/29; 435/6

(57) ABSTRACT

The present invention relates to methods and means for ammonia and/or ammonium transport in a variety of organisms, including mammals, yeast and plants. In particular, the present invention is related to the use of isolated polypeptide molecules, which are particular members of the aquaporin superfamily, and isolated nucleic acid molecule that encode such polypeptides in the transport of NH₄⁺/NH₃ across a membrane.

The present invention relates to methods and means for ammonium and ammonia transport in a variety of organisms, in particular to ammonium and ammonia transport in mammals and plants.

### BACKGROUND ART

The ammonium ion (NH₄⁺) and its conjugated base ammonia (NH₃) are the primary substrates for the synthesis of amino acids, essential to all living cells and can accumulate to millimolar levels within cells. In plants, inefficient recycling and storage of NH₄⁺/NH₃ leads to reduced nitrogen utilisation, sub-optimum growth and may cause significant loss of NH₃ to the atmosphere, thereby resulting in atmospheric pollution¹.

In humans, high levels of extra-cellular NH₄⁺/NH₃ inhibit insulin release^{2, 3}, cause metabolic acidosis and renal failure^{4,5}, and can result in central nervous system dysfunction (leading to Alzheimer's disease⁶ and hepatic encephalopathy⁷).

In animals, NH₄⁺/NH₃ influx into cells has been previously reported to occur via the Na⁺—K⁺-ATPase⁴ or Na⁺—NH₄⁺—2Cl⁻ co-transporter⁵.

Bacteria, yeast and plants have ammonium transporters belonging to the AMT/MEP (methylamine permease) family. These transporters are so-called high-affinity transporters, where transport capacity saturates at concentrations above 100 μM NH₄^{8, 9, 10, 11}. AMT/Mep transporters are carrier type transporters where the transport of NH₄⁺ is energized by the membrane potential¹². Transport of NH₄⁺ through AMT/Mep transporters is therefore limited towards compartments with a negative membrane potential. In turn this will limit the application and use of such transporters.

An abstract discussing certain Tonoplast Intrinsic ProteinS (TIPs) was presented by the present inventors at the XXI Congress of the Scandinavian Plant Physiology Society held 21-24 Aug. 2003. However this did not disclose the sequences, detailed properties, or structure\function relationships disclosed in the present application, which properties and relationships have important implications for the use of particular classes of ammonia transporting proteins.

### DISCLOSURE OF THE INVENTION

The present inventors have identified an ammonium/ammonia specific transport by members of the aquaporin superfamily, constituting channels from plants and mammals.

Specifically, the present inventors identified, using functional complementation in yeast (31019b; Mata, ura3, mep1Δ, mep2Δ::LEU2, mep3Δ::kanMX2⁸), three complementary DNAs (cDNAs) from Triticum aestivum with open reading frames of 747 bp coding for 248-amino acid proteins of TIP2 homologues (Tonoplast Intrinsic Protein). Sequences have been submitted to the NCBI database and are referred to as AY525639, AY525640 and AY535641 for Ta TIP2;1, Ta TIP2;2 and Ta TIP2;3 respectively. Tonoplast intrinsic proteins (TIPs) were previously identified as members of the aquaporin superfamily¹³. TIPs were subsequently classified according to sequence similarity into TIP1-TIP5 (in Arabidopsis).

In the functional cloning approach, a Triticum aestivum cDNA library was transformed into a Saccharomyces cerevisiae mutant that grows poorly on media in which 5 mM NH₄⁺ is the sole nitrogen source. This resulted in the isolation of the Ta TIP2 cDNAs, which restored the ability of the S. cerevisiae mutant to grow normally when 2 mM NH₄⁺ was the sole nitrogen source.

These cDNA sequences were then used as the basis of database searches, which revealed homology with the superfamily of aquaporins, which are known as water transporting proteins. Some aquaporins have also been shown to be involved in transport of glycerol and urea¹⁴.

The cDNA sequences identified by the present inventors show no sequence similarity to the AMT/MEP(methylamine permease) ammonium transporters in bacteria, yeast and plants^{8, 9, 10, 11}.

The inventors then subcloned cDNAs from several different aquaporin homologues into the yeast expression vector pYES2, expressed them in yeast 31019b and showed that in addition to Ta TIP2s, also At TIP2;1 and Hs AQP8 restore the growth of the yeast mutant Δmep1-3 when NH₄⁺ was the sole nitrogen source.

Controversially, it has been suggested¹⁵, that human aquaporin 1 (AQP1) might facilitate diffusion of NH₄⁺/NH₃ although water transport is it's primary function. However, Hs AQP1 failed here to transport NH₄⁺/NH₃. Homology modelling and functional characterization of different aquaporins led to the observation that substitutions of residues in the constriction region may be critical to allow NH₄⁺/NH₃ transport through aquaporins. In particular, substitution of H182 and C191 in Hs AQP1 into smaller and more hydrophobic residues seemed to be needed to allow the transport of NH₄⁺/NH₃. Plant TIP2 and mammalian AQP8 isoforms show isoleucine and glycine substitutions in the respective positions. The inventors then showed that substituting I184 and G193 by histidine and cysteine, the respective residues in human AQP1, completely abolished NH₃/NH₄⁺ transport when expressed in yeast.

The inventors also demonstrated the functional characteristics of aquaporin proteins from plants, humans and mice by expressing these proteins in Xenopus oocytes. Addition of NH₄⁺ to Xenopus oocytes resulted in a continuous acidification of the medium, in line with the interpretation that NH₃ diffused into the oocyte, leaving H⁺ in the external medium. Acidification was significantly increased after injection with Ta TIP2, Hs AQP8, Hs AQP9 and Rn AQP3 mRNA compared to control oocytes injected with water. Expression of human AQP1 did not increase NH₄⁺ induced acidification compared to water injected controls although water transport could be demonstrated for both Ta TIP2;1, Hs AQP8, Rn AQP3, Hs AQP9 and Hs AQP1 mRNA-injected oocytes (Table 1).

Voltage clamp studies on oocytes expressing NH₄⁺/NH₃ transporting aquaporin homologues revealed that at elevated NH₄⁺/NH₃ concentrations a current was associated with the transport of NH₄⁺/NH₃. Yet, the conduction was not different with 20 mmol l⁻¹ of NH₄⁺ at pHₑ 7.4, 10 mmol l⁻¹ NH₄⁺ at pHₑ 7.7, or 5 mmol l⁻¹ at pHₑ 8.0, experiments where the H⁺ concentration decreases while the NH₃ concentration remains constant. The inventors therefore conclude that the transport of NH₄⁺ through these channels is dependent on NH₃.

In addition to TIPs and Hs AQP8, also Rn AQP3 and Hs AQP9 transported NH₄⁺/NH₃ when expressed in Xenopus oocytes. In all these isoforms, amino acid residues lining the constriction region differ from the residues in AQP1, in line with the interpretation that substitutions in the constriction region are critical for NH₄⁺/NH₃ transport through aquaporin homologues.

The transporter proteins identified by the present inventors show both NH₄⁺/NH₃ specificity and bidirectional transport, the latter evidenced by the fact, that yeast expressing Ta TIP2 displays a growth disadvantage over yeast transformed with an empty vector when grown on alternative N-sources such as arginine and proline and a relatively high pH (pH 7.5). At these conditions, NH₄⁺/NH₃ generated in the yeast by deamination of the amino acids is secreted into the medium via the TIP2 channel.

### DETAILED DISCLOSURE OF INVENTION

Various aspects of the present invention will now be discussed in more detail:

At its most general, the present invention relates to a particular class of isolated polypeptides which are members of the aquaporin superfamily, or derivatives thereof, and their use NH₃/NH₄⁺ transporters e.g. to influence cellular pH homeostasis. As shown in the examples below, preferred transporters may be both specific and high-capacity. It further relates to isolated nucleic acid molecules, which encode such transporters. The invention provides, inter alia, a method of influencing or affecting NH₃/NH₄⁺ transport across a membrane by introducing such a heterologous transporter into the membrane.

The “membrane” may or may not be part of a cell, such as a plant, yeast or mammalian cell. The use of artificial membranes is discussed further below.

NH₄⁺/NH₃ specific transporter activity may be assessed using tracer techniques, which are described in more detail below (Example 5).

Preferably, the NH₃-transporter is a bidirectional NH₄⁺/NH₃ transporter. In contrast to the transport of NH₄⁺ through AMT/Mep transporters, the direction of transport through NH₄⁺/NH₃ transporting aquaporin homologues is regulated by both the concentration of NH₄⁺/NH₃ and the pH of the compartments surrounding the membrane. Thus preferably the transport is NH₄⁺, NH₃ and H⁺ dependent i.e. may be driven by a concentration gradient of any of these things across a membrane.

Bidirectional and gradient dependent transport may be assessed using simultaneous measurements of efflux and uptake of different N isotope labelled NH₄⁺/NH₃, for examples using either yeast or Xenopus oocytes expressing the transporter.

### Aquaporin Superfamily

By “aquaporin superfamily” is meant all naturally occurring homologues of the sequences shown in FIG. 1. Such proteins are characterised by having six predicted membrane-spanning domains and two characteristic conserved NPA/V motifs within a membrane embedded loop following membrane-spanning domains two and four respectively (FIG. 3). Members of the aquaporin superfamily will generally have at least 22% identity at the amino acid level with the TIP2 amino acid sequence shown in FIG. 1. Aquaporin superfamily nucleic acids encode these polypeptides.

TIP2s form a sub-group of the super family of aquaporins in plants, which have been localized to membranes of vacuoles specialized for storage of proteins in plants^{16, 17}. However, TIP2s have recently also been localized to the peribacteroid membrane surrounding nitrogen fixating bacteroids in legume plant¹⁸.

The polypeptides of the present invention are those which have a characteristic constriction region shown by the present inventors to provide advantageous properties. This region is defined by residues F58, H182, C191 and R197 in bovine AQP1. The equivalent residues can be identified in other AQPs without burden by those skilled in the art—see for example FIG. 1, or FIG. 6B.

The constriction region will be constituted by residues different to those in natural AQP1 homologues i.e. will not have all of the residues given above, and will preferably not have H182 and C191.

Preferably the constriction region is constituted by the following residues: 182 and 191. Preferably in place of H182 (numbering according to bovine AQP1) the constriction region will comprise isoleucine, valine or a small residue such as glycine and alanine. In place of C191 it may comprise glycine, alanine or a tyrosine.

Homology (e.g., similarity or identity) may be as defined using sequence comparisons made using FASTA and FASTP¹⁹. Parameters are preferably set, using the default matrix, as follows: Gapopen (penalty for the first residue in a gap): −12 for proteins/−16 for DNA; Gapext (penalty for additional residues in a gap): −2 for proteins/−4 for DNA; KTUP word length: 2 for proteins/6 for DNA. Homology may be at the nucleotide sequence and/or encoded amino acid sequence level.

As discussed hereinafter, further naturally occurring members of the aquaporin superfamily may be identified, using the members of the aquaporin superfamily members, which are described above, e.g., by using the sequence of Hs AQP3, 8, 9, At TIP2;1, Ta TIP2;1 or fragments thereof, or antibody screening. Preferred sources from which the aquaporin polypeptide or nucleic acid molecule may be derived include: human; Mus musculus (mouse); S. cerevisiae; Triticum aestivum (wheat); Arabidopsis thaliana.

Human or animal aquaporins, in particular AQP3, 8 and 9 may be preferred for the therapeutic embodiments of the present invention discussed in more detail below.

### Derivatives of Aquaporin Superfamily

It will be understood that in the various aspects of the invention, derivatives or other variants of any of the members of the aquaporin superfamily may be used in the context of NH₄⁺/NH₃ transport. Such derivatives may be produced, e.g. by site directed or random mutagenesis, or by direct synthesis.

For example, a variant or derivative nucleic acid molecule may share homology with, or be identical to all or part of one of the coding sequences of a nucleotide sequence of the invention discussed herein. Preferably, the nucleic acid and/or amino acid sequence shares at least about 60%, or 70%, or 80% homology, most preferably at least about 90%, 95%, 96%, 97%, 98% or 99% homology with one of the NH₄⁺/NH₃ transporter sequences disclosed herein.

Generally variants or derivatives may be (or encode, or be used to isolate or amplify nucleic acids which encode) polypeptides that are capable of transporting NH₄⁺/NH₃ and/or which will bind specifically to an antibody raised against one of the polypeptides shown in FIG. 1. NH₄⁺/NH₃ transport may be assessed as described above.

Thus a variant or derivative may be a distinctive part or fragment (however produced) corresponding to a portion of the sequence provided. The fragments may be (or encode) particular functional parts of the polypeptide. Equally the fragments may have utility in probing for, or amplifying, the sequence provided or closely related ones.

In one aspect the invention provides a process for enhancing the NH₄⁺/NH₃ transport properties of an AQP (for example those preferred properties describes above) which method comprises modifying the constriction region residues to those preferred residues described above e.g. small hydrophobic residues.

For brevity, hereinafter, the term “aquaporin polypeptide” (or “aquaporin nucleic acid” molecule, as appropriate) encompasses any of the members of the aquaporin family described or identified as described above, and derivatives thereof, in each case having the characteristic constriction region defined above.

### Modes of Use

Since transport of NH₄⁺/NH₃ through aquaporins is bidirectional, the TIP2-like proteins may be used to alleviate stress or disease conditions characterized by both high levels of extracellular NH₄⁺/NH₃, as well as high levels of cytoplasmic NH₄⁺/NH₃. In humans, high levels of extracellular NH₄⁺/NH₃ inhibit insulin release^{2, 3}, cause metabolic acidosis and renal failure^{4, 5}, and can result in central nervous system dysfunction (leading to Alzheimer's disease⁶ and hepatic encephalopathy⁷. In plants, TIP2-like proteins may be used for handling elevated cytoplasmic NH₄⁺/NH₃, by facilitating its transport into intracellular storage compartments, which in turn can lead to improved nitrogen fertilizer utilization and environmental stress tolerance.

Where polypeptides and nucleic acid molecules are used in accordance with the present invention they may be provided isolated and/or purified from their natural environment, in substantially pure or homogeneous form, or free or substantially free of other nucleic acids of the species of origin. Where used herein, the term “isolated” encompasses all of these possibilities.

Nucleic acid molecules may be wholly or partially synthetic. In particular they may be recombinant in that nucleic acid sequences, which are not found together in nature (do not run contiguously) have been ligated or otherwise combined artificially. Alternatively they may have been synthesised directly e.g. using an automated synthesiser. Nucleic acid used according to the present invention may include cDNA, RNA and modified nucleic acids or nucleic acid analogues. Where a DNA sequence is specified, e.g. with reference to a figure, unless context requires otherwise the RNA equivalent, with U substituted for T where it occurs, is encompassed. Where a nucleic acid (or nucleotide sequence) of the invention is referred to herein, the complement of that nucleic acid (or nucleotide sequence) will also be embraced by the invention. The ‘complement’ in each case is the same length as the reference, but is 100% complementary thereto whereby by each nucleotide is capable of base pairing with its counterpart i.e. G to C, and A to T or U.

### OTHER ASPECTS AND EMBODIMENTS

In addition to the uses discussed above, the invention further provides a method of transporting NH₄⁺/NH₃ across a membrane using a member of the aquaporin superfamily as described above. Such a method may comprise the use of any aquaporin polypeptide or nucleic acid molecule as discussed herein.

For example, such a method may comprise incorporating an aquaporin polypeptide into a membrane, and exposure of the membrane to NH₃/NH₄⁺ ions.

The transporter protein may be inserted into artificial membranes using the standard technique of reconstitution of the protein into artificial membranes.

Alternatively, such a method may comprise partial purification of a membrane comprising an aquaporin as described herein and exposure of the membrane to NH₃/NH₄⁺ ions.

Such a method may alter existing NH₄⁺/NH₃ transport across a membrane (e.g., may influence or affect the nature or degree of such transport, in particular in respect of the properties discussed above), or may impart NH₃/NH₄⁺ transport on a membrane which previously had no such capacity.

### Use of Aquaporin Nucleic Acids

The polypeptide of the aquaporin superfamily may be provided by expression from an isolated nucleic acid molecule as described herein. Suitable expression systems are discussed in further detail below.

Preferably the isolated nucleic acid molecule for such use comprises a sequence, which encodes an amino acid sequence shown in FIG. 1, more preferably, the isolated nucleic acid molecule comprises a nucleotide sequence deposited as described above.

Thus the present invention provides the use of an isolated nucleic acid molecule encoding an aquaporin polypeptide, in influencing or affecting (e.g., enhancing) NH₄⁺/NH₃ transport across a membrane.

Where the membrane is part of a cell, the polypeptide may be inserted into the membrane of the cell following expression from an encoding nucleic acid (e.g. as present in a vector) as described in more detail below.

### Use of Vectors

The nucleic acids encoding the NH₄⁺/NH₃ transporters for use in the various aspects of the invention may be in the form of a recombinant and preferably replicable vector.

Such a ‘vector’ may be any plasmid, cosmid, or phage in double or single stranded linear or circular form which may or may not be self transmissible or mobilizable, and which can transform prokaryotic or eukaryotic host either by integration into the cellular genome or exist extrachromosomally (e.g. autonomous replicating plasmid with an origin of replication).

Generally speaking, those skilled in the art are well able to construct vectors and design protocols for recombinant gene expression. For further details see references^{20, 21}, which are incorporated herein by reference.

### Transformed Cells and Organisms

In one embodiment there is provided the use of a nucleic acid molecule, which encodes an aquaporin polypeptide in influencing or affecting NH₄⁺/NH₃ transport in a cell e.g. yeast, plant, or mammalian cell.

Such a nucleic acid molecule may comprise further sequences, in addition to a sequence encoding an aquaporin polypeptide, encoding one or more signal peptides for insertion of the protein into the appropriate membrane. Signal sequences are discussed in more detail later.

Where a nucleic acid molecule is expressed in a cell from a heterologous gene, the heterologous gene may replace an endogenous equivalent gene, i.e. one, which normally performs the same or a similar function, or may be additional to an endogenous gene or other sequence.

Accordingly, the invention further provides a method of influencing or affecting the nature or degree of NH₄⁺/NH₃ transport in a cell, comprising the step of causing or allowing expression of a heterologous nucleic acid sequence as discussed above within the cell.

The AQPs discussed herein may be used markers for the selection of transgenic cells, or as markers e.g. in breeding technology.

The cell may be in an organism (e.g. plant or mammal) in order to influence or affect the nature or degree of NH₄⁺/NH₃ transport in that organism. It should be noted that the nucleic acids may be used to both enhance and down-regulate NH₄⁺/NH₃ transport (as discussed below).

The present invention further provides a method of producing an NH₄⁺/NH₃ transporter in a cell, comprising the step of causing or allowing expression of a heterologous aquaporin nucleic acid sequence as discussed above within the cell.

Some particular utilities will now be discussed with respect to preferred cells, organisms and vectors.

### Microorganisms

Nucleic acid may be expressed in bacteria, preferred vectors include plasmid, virus or phage vectors provided with an origin of replication, optionally a promoter for the expression of the said polynucleotide and optionally a regulator of the promoter. Such vectors may include a signal sequence to direct the protein so that it is expressed on the cell surface, or is secreted from the cell. Examples of such signal sequences include: outer membrane proteins, for example the OmpA signal peptide; exotoxins, for example exotoxin A from P. aeruginosa. Further examples are described in^{22, 23, 24}.

For transformation into bacterial cells, calcium chloride transformation, electroporation or any other suitable technique may be used. Such techniques are well known to the person skilled in the art and details of exemplary techniques may be found in reference²⁰.

Preferred vectors for expression in yeast cells include pYES2, pFL61 and pYC2, and, standard transformation techniques include electroporation and heat-shock.

Transformed recombinant bacteria (e.g., E. coli) or yeast cells (e.g., S. cerevisiae) over-expressing the NH₄⁺/NH₃ transporter may be useful sources of NH₄⁺/NH₃ transporter for a variety of uses, or may be used as a source of sense or anti-sense RNA, or of nucleic acids for use in gene therapy.

Following expression, the recombinant product may, if required, be isolated from the expression system.

### Plants

Where the present invention is applied to plants, transgenic plants may be generated which over-express an NH₄⁺/NH₃ transporter as described herein, to increase plant growth, crop productivity and nitrogen utilisation efficiency; to increase crop yield and tolerance to abiotic and biotic stress factors; to minimise the consumption of fertilisers and reduce losses of nitrogen to the environment; to increase plant stress tolerance towards elevated temperature and light intensities or to increase plant stress tolerance towards plant pathogens or herbicides; to alter tolerance to NH₄⁺ or NH₃ applies to the environment of the plant e.g. by foliar spraying with inorganic or organic nitrogen solutions. Such transgenic plants may have utility in screening for herbicides which affect NH₄⁺/NH₃ transport.

Alternatively, the NH₄⁺/NH₃ transporter may be expressed in cell or organelle membranes so that the NH₄⁺ produced in various metabolic processes is appropriately transported within the cell to the right places in the cell in order to be efficiently re-assimilated. In such a situation, a signal peptide may be used to appropriately target the protein, e.g., for appropriate targeting to chloroplastic, mitochondrial and vacuolar membranes.

In another way, the NH₄⁺/NH₃ transporter may be expressed or repressed in the leaves to minimise the volatilisation of NH₃. In periods of high light intensity and air temperature, photorespiration causes generation of large quantities of NH₄⁺ in the mitochondria, which after conversion to NH₃ can be lost into the atmosphere. The process is known as NH₃ volatilisation and is a source of atmospheric pollution.

In a further approach, transgenic legumes may be produced which overexpress the transporter protein in the root nodules, in order to maximise the benefit to the plant of the NH₄⁺ produced by symbiotic fixation of atmospheric nitrogen by the Rhizobia bacteria living in the root nodules.

Where nucleic acid is expressed in a plant cell or plant, exemplary procedures and vectors are described²⁵. For example, suitable promoters include the Cauliflower Mosaic Virus 35S (CaMV 35S promoter); and the senescence-specific SAG12 promoter²⁶. Other examples are disclosed in²⁷.

The promoter may be selected to include one or more sequence motifs or elements conferring developmental and/or tissue-specific regulatory control of expression. Inducible plant promoters include the ethanol-induced promoter²⁸. It may be desirable to use a strong constitutive promoter such as the ubiquitin promoter, particularly in monocots.

If desired, selectable genetic markers may be included in the construct, such as those that confer selectable phenotypes such as resistance to antibiotics or herbicides (e.g. kanamycin, hygromycin, phosphinotricin, chlorsulfuron, methotrexate, gentamycin, spectinomycin, imidazolinones and glyphosate).

Nucleic acid can be transformed into plant cells using any suitable technology, such as a disarmed Ti-plasmid vector carried by Agrobacterium exploiting its natural gene transfer ability (EP-A-270355, EP-A-0116718, NAR 12(22) 8711-87215 1984), particle or microprojectile bombardment (U.S. Pat. No. 5,100,792, EP-A-444882, EP-A-434616) microinjection (WO 92/09696, WO 94/00583, EP 331083, EP 175966, Green et al. 1987, Plant Tissue and Cell Culture, Academic Press), electroporation (EP 290395, WO 8706614 Gelvin Debeyser) other forms of direct DNA uptake (DE 4005152, WO 9012096, U.S. Pat. No. 4,684,611), liposome mediated DNA uptake²⁹, or the vortexing method³⁰. Physical methods for the transformation of plant cells are reviewed in³¹.

Generally speaking, following transformation, a plant may be regenerated, e.g. from single cells, callus tissue or leaf discs, as is standard in the art. Almost any plant can be entirely regenerated from cells, tissues and organs of the plant. Available techniques are reviewed in^{32, 33}. The generation of fertile transgenic plants has been achieved in the cereals rice, maize, wheat, oat, and barley^{34, 35, 36, 37}.

Thus the invention further provides a method of influencing or affecting the NH₄⁺/NH₃ transport in a plant (e.g. to affect the properties of the plant as described above) which method includes the step causing or allowing expression of a heterologous nucleic acid sequence as discussed above within the cells of the plant.

The step may be preceded by the earlier step of introduction of the nucleic acid into a cell of the plant or an ancestor thereof.

### Mammals

The following cell systems are examples of those which may be used where expression in mammalian cells is desired, e.g.: COS, CHO, BHK, 293, 3T3. However, any suitable expression construct may be used as would be understood by the person skilled in the art. For example, a suitable expression construct may comprise a promoter derived from the genome of mammalian cells (e.g., metallothionein promoter) or from mammalian viruses (e.g., the adenovirus late promoter; the vaccinia virus 7.5K promoter). Suitable expression systems include viral-based expression systems, e.g., based on adenovirus; or pXT1, pS65, or p3′SS expression vectors.

Mammalian cells may be transfected by any suitable technique such as lipofection or standard calcium phosphate chloride method. DNA may be incubated in HEPES buffered saline and precipitated using calcium chloride, followed by incubation at room temperature for, e.g. 20 minutes. The precipitated DNA is then added to cells, which are then incubated at room temperature before addition of medium/FCS for overnight incubation.

In mammals, such as humans, transporter nucleic acid molecules and polypeptides may be utilised to limit metabolic acidosis in the kidney, which results from increased levels of NH₄⁺, or to avoid central nervous system dysfunction, Alzheimer Type II astrocytosis and brain oedema, which result from hyperammonaemia. Other utilities are discussed below. Drugs may be identified or designed which manipulate (e.g., increase or decrease the activity of the transporter protein.

### Methods of Treatment

Where the use of the isolated nucleic acid molecule is applied to mammals (especially humans), the nucleic acids or polypeptides may be for use in a method of treatment for a disorder associated with NH₄⁺ e.g. high levels of NH₄⁺.

Preferred AQPs for use in this aspect include human or animal AQP3, 8 or 9.

Therefore, the invention also encompasses the nucleic acids or polypeptides disclosed herein for use in a method of treatment for a disorder associated with high levels of NH₄⁺.

The invention further encompasses the use of the nucleic acids or polypeptides disclosed herein in the manufacture of a medicament for the treatment or prophylaxis of a disorder associated with high levels of NH₄⁺. Such a medicament may further comprise a suitable excipient or carrier.

Methods of treatment of a disorder associated with high levels of NH₄⁺ also form a further aspect of the invention, such methods may comprise administering a nucleic acid molecule or polypeptide as described herein to an individual.

Disorders associated with high levels of NH₄⁺ include, but are not limited to metabolic acidosis in the kidney, central nervous system dysfunction, Alzheimer's Type II astrocytosis, and brain oedema.

For example, the nucleic acids of the invention may be administered in a form of gene, cell or tissue therapy to a patient.

For example, in a method of gene therapy one or more copies of a nucleic acid sequence as described herein (e.g., a aquaporin family member such as a sequence encoding one of the sequence shown in FIG. 1, or variants thereof) may be inserted into the appropriate cells within a patient, using vectors that include, but are not limited to adenovirus, adeno-associated virus, and retrovirus vectors, in addition to other particles that introduce DNA into cells, such as liposomes. The person skilled in the art is readily able to produce such a gene therapy vector. For an example see, Anderson, U.S. Pat. No. 5,399,349.

Such gene therapy vectors may incorporate targeting signals to the appropriate membrane or organ. Alternatively, or additionally cell or organelle specific promoters may be used.

In a method of cell or tissue therapy, the living therapeutical cells or tissues containing the nucleic acid sequence as described herein, or copies thereof, are implanted in the patient.

### Drug Screening

The aquaporin polypeptides as disclosed herein may be used purified, isolated or in-vivo for screening of low molecular weight compounds affecting their activity and or expression level, directly or indirectly e.g. in a method for screening for medicaments/drugs against the disorders discussed herein.

The promoter used in connection with a reporter gene for the screening of putative effectors of gene expression of members of the aquaporin family.

Antibodies, Peptides, Proteins and/or Polymers

Purified or isolated aquaporin polypeptides as disclosed herein, e.g., produced recombinantly by expression from encoding nucleic acid therefore, may be used to raise antibodies employing techniques, which are standard in the art.

Such antibodies may be used in a method of influencing or affecting the NH₃/NH₄⁺ transport in a cell or organism, and accordingly the use of an antibody which binds a aquaporin-polypeptide in influencing or affecting NH₄⁺/NH₃ transport across a membrane represents a further aspect of the invention.

Such antibodies may be for use in the treatment of a disorder associated with high levels of NH₄⁺ ion, and the use of such antibodies in the manufacture of a medicament for the treatment or prophylaxis of such a disorder, and a method of treatment or prophylaxis of such a disorder comprising administering such an antibody to an individual, represent further aspects of the invention.

Methods of producing antibodies include immunising a mammal (e.g. mouse, rat, rabbit, horse, goat, sheep or monkey) with the protein or a fragment thereof. Antibodies may be obtained from immunised animals using any of a variety of techniques known in the art, and might be screened, preferably using binding of antibody to antigen of interest. For instance, Western blotting techniques or immunoprecipitation may be used³⁸.

### Methods of Identifying NH₄⁺ Specific Transporters

In a further aspect of the invention, there is provided a method of identifying and/or cloning, from a eukaryotic cell, a nucleic acid molecule encoding a NH₄⁺/NH₃ transporter (such as those having enhanced the NH₄⁺/NH₃ transport properties described above), which method employs a nucleic acid molecule encoding a aquaporin polypeptide (e.g., uses a sequence described herein or a derivative thereof, such as a fragment, or complementary sequence). Eukaryotic cells, which may be used in the cloning include plant cells, yeast cells, mammal cells.

In one aspect the invention provides such a method of identification, which method comprises selecting sequences encoding the preferred constriction region residues described above e.g. small hydrophobic residues.

In a further aspect the present invention provides an isolated nucleic acid molecule identified or cloned by such a method.

For example methods of cloning or identification may involve using an oligonucleotide in probing or amplification reactions (e.g., PCR) comprising or consist of a distinctive sequence of about 48, 36 or fewer nucleotides in length (e.g. 18, 21 or 24). Generally specific primers are upwards of 14 nucleotides in length. For optimum specificity and cost effectiveness, primers of 16-30 nucleotides in length (which sequence is not present in genes of the prior art) may be preferred. Preferably the sequence will include codons encoding all or part of the constriction region e.g. at least 2 residues thereof.

Probing may employ any standard technique. Those skilled in the art are well able to employ suitable conditions of the desired stringency for selective hybridisation, taking into account factors such as oligonucleotide length and base composition, temperature and so on. One common formula for calculating the stringency conditions required to achieve hybridization between nucleic acid molecules of a specified sequence homology is (Sambrook et al., 1989): Tₘ=81.5° C.+16.6 Log [Na⁺]+0.41 (% G+C)−0.63 (% formamide)-600/#bp in duplex. As an illustration of the above formula, using [Na⁺]=[0.368] and 50-% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C. Such a sequence would be considered substantially homologous to the nucleic acid sequence of the present invention.

Alternatively, antibodies raised to a polypeptide or peptide (antibodies are discussed in more detail below) can be used in the identification and/or isolation of homologous polypeptides, and then the encoding genes.

Thus, a method of identifying or isolating a polypeptide (which include novel polypeptides) with NH₄⁺/NH₃ transporter function may comprise screening candidate peptides or polypeptides with a polypeptide including the antigen-binding domain of an antibody (for example whole antibody or a fragment thereof) which is able to bind an NH₄⁺/NH₃ transporter peptide as disclosed herein, or fragment, or variant thereof or preferably has binding specificity for such a peptide or polypeptide, such as having an amino acid sequence identified herein.

Candidate peptides or polypeptides for screening may for instance be the products of an expression library created using nucleic acid derived from cells of interest, or may be the product of a purification process from a natural source.

### Inhibition of NH₄⁺/NH₃ Transport

The foregoing discussion has been generally concerned with uses of the nucleic acids of the present invention for production of functional polypeptides, thereby increasing the NH₄⁺/NH₃ transport activity in the cell.

However the information disclosed herein may also be used to reduce the activity in cells in which it is desired to do so.

For instance down-regulation of expression of a target gene may be achieved using anti-sense technology.

Antisense technology is reviewed in^{39, 40}. The complete sequence corresponding to the coding sequence (in reverse orientation for anti-sense) need not be used. For example fragments of sufficient length may be used. It is a routine matter for the person skilled in the art to screen fragments of various sizes and from various parts of the coding sequence to optimise the level of anti-sense inhibition. It may be advantageous to include the initiating methionine ATG codon, and perhaps one or more nucleotides upstream of the initiating codon. A further possibility is to target a conserved sequence of a gene, e.g. a sequence that is characteristic of one or more genes, such as a regulatory sequence.

An alternative to anti-sense is to use a copy of all or part of the target gene inserted in sense, that is the same, orientation as the target gene, to achieve reduction in expression of the target gene by co-suppression. See, for example^{41, 42, 43}, and U.S. Pat. No. 5,231,020.

Further options for down regulation of gene expression include the use of ribozymes, e.g. hammerhead ribozymes, which can catalyse the site-specific cleavage of RNA, such as mRNA (see e.g.^{44, 45}).

Thus, inter alia, the use of aquaporin nucleotide sequences, which are complementary to any of those, coding sequences, disclosed above, for such down regulation of transport activity forms one part of the present invention.

The invention will now be further described with reference to the following non-limiting examples. Other embodiments of the invention will occur to those skilled in the art in light of these.

### FIGURES & TABLE

FIG. 1 shows an amino-acid sequence alignment of aquaporins from Triticum aestivum, Arabidopsis thaliana, Saccharomyces cerevisiae, E. coli, Bos taurus and humans. Residues identical to Hs AQP1 are shaded black. The overall consensus is shaded grey.

FIG. 2 shows an aquaporin superfamily phylogenetic tree including sequences from Triticum aestivum, Arabidopsis thaliana, Saccharomyces cerevisiae, Echerichia coli and humans (maximum parsimony).

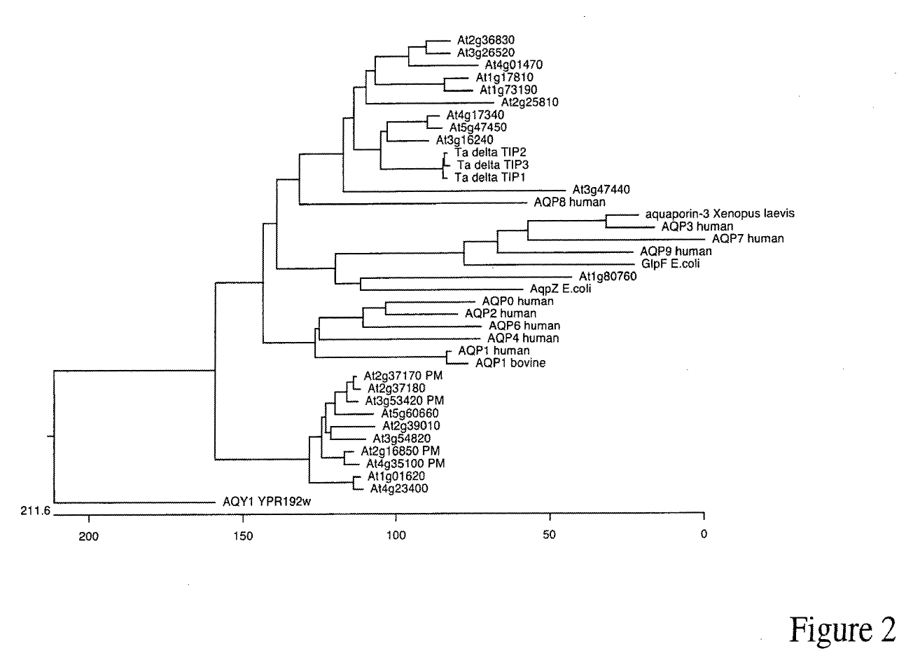

FIG. 3 shows a Kyte-Doolittle hydrophobicity plot of TIP2;1 from wheat generated using a 13-amino-acid window. Bars at the bottom of the figure indicate the six membrane spanning domains. A structural presentation is shown below.

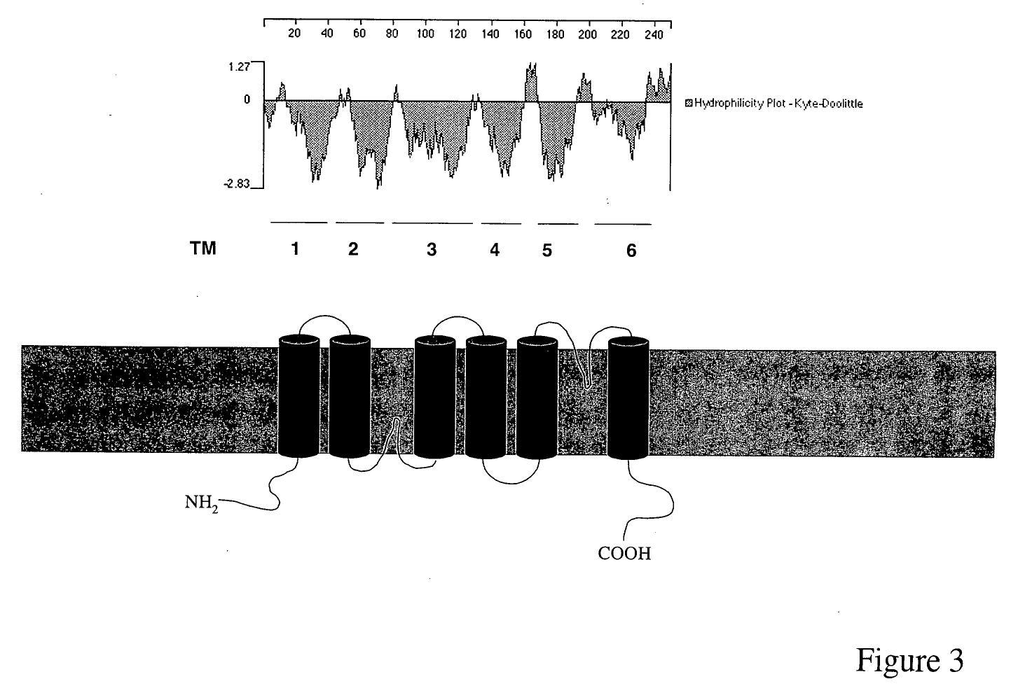

FIG. 4 shows the complementation of the yeast mutant Δmep1-3 by high affinity ammonium transporters (Ta AMTs) and different aquaporins (Ta TIP2s). Control (pYES2) is the yeast transformed with an empty pYES2. Ta TIP2s are the wheat aquaporins. (A) The yeasts were grown on media containing galactose and either 0.1% proline or different concentrations of NH₄⁺ as indicated. The pH of the medium was 5.5. (B) Yeast growth was tested at different pH of the medium as indicated in the figure.

FIG. 5 illustrates the structural model of Ta TIP2;1 as compared to the structure of bovine AQP1. (A) Longitudinal view; bovine AQP1 (black) and homology model of Ta TIP2;1 (grey). The highly conserved NPA (asparagine, proline, alanine) signature motifs are shown in yellow. (B) View through the channel pore from the cytoplasmic face; residues from bovine AQP1 are in front and labeled. (C) View from the extra-cytoplasmic face; residues from Ta TIP2;1 are in front and labeled. The position of the water molecule coordinated by H182 and the carbonyl oxygen of G192 in the structure of AQP1 is included (B and C).

FIG. 6 illustrates the results of the functional complementation of the yeast mutant expressing from the multi-copy vector pYES2 either different aquaporin homologues or no protein (pYES2). The different yeast strains were grown on galactose containing medium supplemented with either proline or different concentrations of NH₄⁺ as the nitrogen source.

FIG. 7 shows growth of yeast transformed with either Ta TIP2;1 (black) or pYES2 (red; control) at pH 7.5 and 0.1% arginine as sole N source. The cultures were inoculated with an equal amount of cells and the optical density (OD 600 nm) was measured at 600 nm and the time points indicated.

FIG. 8 shows results from extracellular pH measurements of the bathing medium containing 20 Xenopus oocytes after injection with either water (control) or Ta TIP2;2 mRNA (mRNA injected). The pH was recorded for 30 minutes either in the presence or absence of NH₄⁺. A, pH of the bathing medium as a function of time; B, pH changes relative to the starting pH.

FIG. 9 illustrates data from time dependent influx measurements of (A) ¹⁴C-methyl ammonium and (B) ¹⁴C-formamide into oocytes either injected with water (control) or Ta TIP2;2 mRNA (mRNA injected). The external concentration of both methyl ammonium and formamide was 20 mM.

FIG. 10 shows the effects of NH₄⁺ on membrane potential Eₘ and volume (V) of AQP8- and AQP1- expressing oocytes compared to native oocytes. The Lₚ of the oocytes was measured by the abrupt addition of 20 mosm l⁻¹ of mannitol (man). After this the effects of the isosmotic addition of 20 mmol l⁻¹ of NH₄Cl at pH of 7.4 was tested (replacing NaCl). This induced rapid and large depolarizations in the membrane potential Eₘ of AQP8-expressing oocytes and slow and small depolarizations in AQP1 expressing and native oocytes.

FIG. 11 shows clamp currents (I_{C}) induced by NH₄⁺ as a function of external pH (pHₑ) in AQP8-expressing and native oocytes. (A) An AQP8 expressing oocyte was clamped to −50 mV, and 5 mmol l⁻¹ of NH₄⁺ was added isosmotically (replacing Na⁺) for 60 sec to the bathing solution (black bars) at four different pHs, 7.1, 7.4, 7.7, and 8.0 (and therefore different NH₃ concentrations). Larger pH gave larger inward clamp currents I_{C}. (B) The same experiments were performed on a native oocyte, which resulted in smaller currents. (C) Clamp currents I_{C} from 5 AQP8-expressing oocytes (open squares) and 5 native oocytes (nat, open circles). The test solutions contained 5 mmol l⁻¹ NH₄⁺ at pHs of 6.8, 7.1, 7.4, 7.7, 8.0, 8.3, or 8.6, the corresponding NH₃ concentrations are given at the abscissa. The difference between the data from the AQP8-expressing oocyte (Mm AQP8) and the data from the native oocyte (filled triangles) was fitted to a sigmoidal function that saturated at around pH 7.7. The Lₚ of the AQP8-expressing oocytes was 7.1±0.81 (5) [10⁻⁵ cm s⁻¹ (osm l⁻¹)⁻¹] and 0.33±0.02 (4) [10⁻⁵ cm s⁻¹ (osm l⁻¹)⁻¹] for the native oocytes. (D) Long term effects of isosmotic application of 5 mmol l⁻¹ of NH₄Cl at pHₑ of 7.4. (E) NH₄⁺ induced clamp currents (I_{C}) in AQP1-expressing and native oocytes as a function of pHₑ as in C.

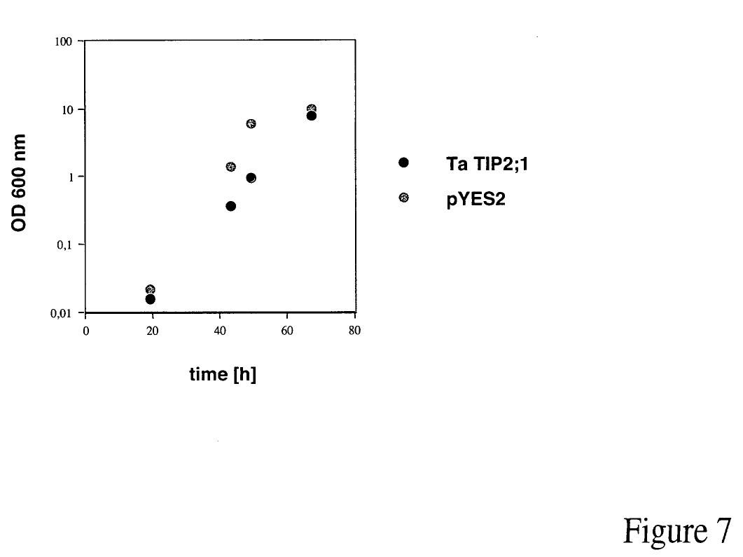

Table 1 shows the initial rates of acidification of the bathing solution of aquaporin-expressing oocytes relative to native oocytes. Experiments as in FIG. 7, units [10⁻¹¹ mol H⁺ sec⁻¹ oocyte⁻¹].

The solutions contained 70 mM of Na⁺ and 20 mM NH₄⁺ and had low buffer capacities (see Methods). They were adjusted to pHₑs of 6.5, 7.4, or 8.5, which gave different NH₃ concentrations. The rates of acidification were calculated as the product of the initial rate of change in pHₑ (see FIG. 7) and the buffer capacity of the bathing solution and given per oocyte. N.S. signifies not significantly different from native oocytes from the same batch. Numbers in parenthesis are number of experiments of 20 oocytes each. The Lₚs for each group of oocytes are given in the lower row [10⁻⁵ cm s⁻¹ (osm l⁻¹)⁻¹]; numbers in parenthesis are that of single oocytes.

### EXAMPLES

### Example 1

### Isolation of Ta TIP2 Genes from Triticum aestivum

A Triticum aestivum cDNA library in pYES2 was transformed into a Saccharomyces cerevisiae mutant (Mata ura3 mep1Δ mep2Δ::Leu2 mep3□::KanMX2) that grows poorly on media with 5 mM NH₄⁺ as the sole nitrogen source. As a result of these functional complementation studies, three different 747-base-pair complementary DNAs (cDNAs) from Triticum aestivum were isolated which restored the ability of the mutant to grow normally when 2 mM NH₄⁺is the sole nitrogen source. Further experimental details relating to functional cloning can be found in¹⁰. These cDNAs include highly similar open reading frames of 747 bp coding for a 248-amino-acid protein, called Ta TIP2; 1-3.

### Example 2

### Identification of Super-Family

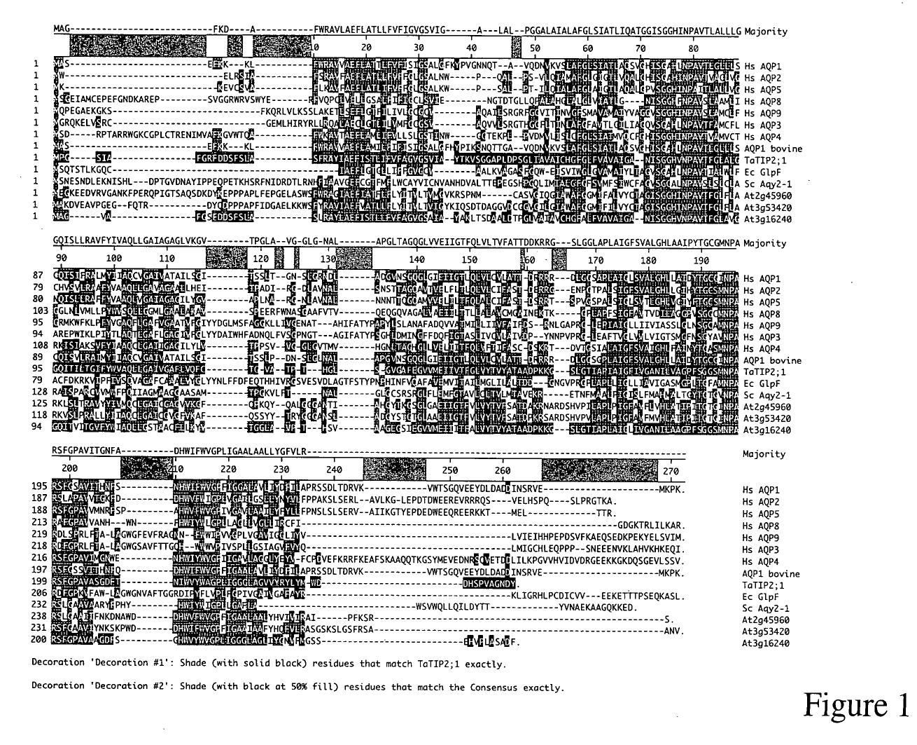

This Ta TIP2 cDNA sequence was then used as the basis of database searches (a BLAST search of GenBank and SwissPROT databases), which revealed a superfamily of highly homologous proteins referred to as aquaporins. This super-family included homologues in all living organisms were sequence information is available. In particular the super-family included 11 isoforms in human called AQP0-AQP10, two isoforms in E. coli (GlpF and AqpZ), 35 sequences in Arabidopsis⁴⁶, and four homologues in the yeast Saccharomyces cerevisiae (Aqy1, Aqy2, Fps1 and YFL054c). Alignments of selected amino acid sequences are shown in FIG. 1.

### Example 3

### Cloning into Yeast Expression Vector

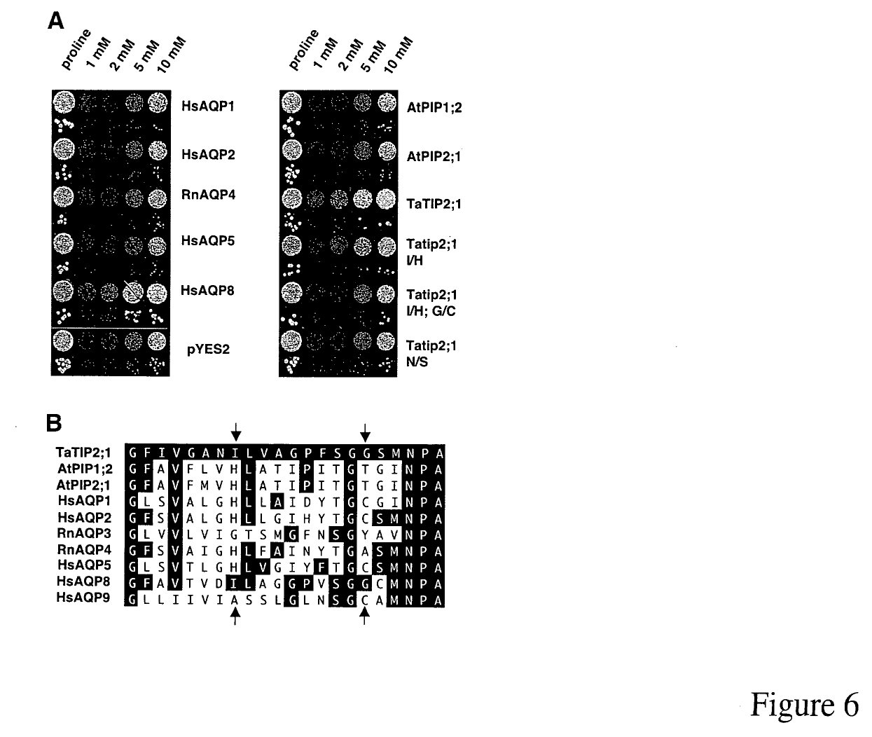

Different mammalian and plant cDNAs, where then subcloned into the yeast expression vector pYES2 and expressed in yeast. In addition to plant TIP2s, AQP8 was shown to significantly improve growth of the yeast mutant when 2 mM NH₄⁺ was the sole nitrogen source. The results of the functional complementation are illustrated in FIG. 6.

### Example 4

### Transport of NH₄⁺/NH₃ into Yeast as Dependent on the Extracellular pH

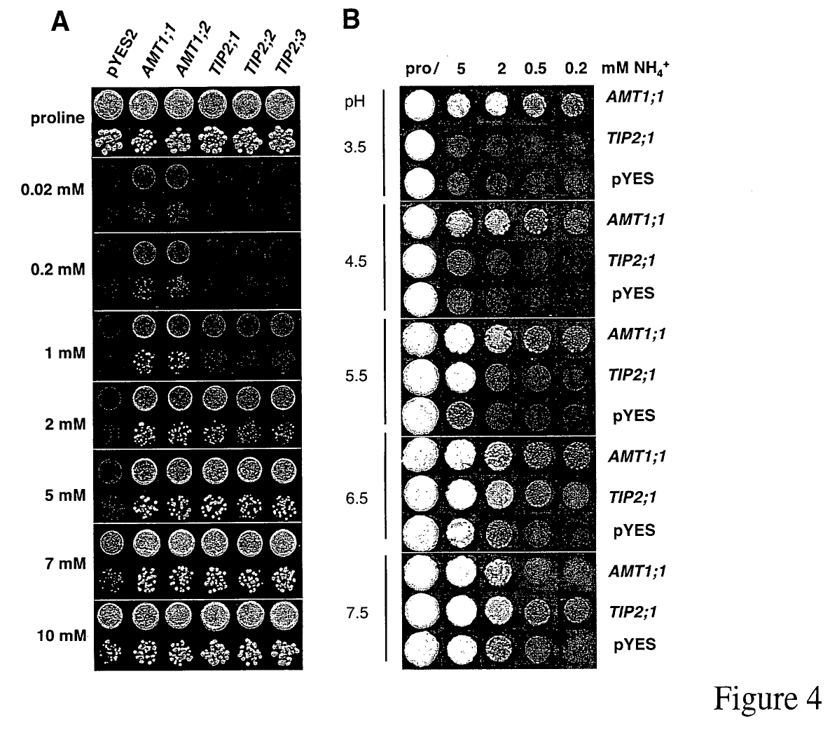

Performances of the yeast mutant Δmep1-3 transformed with either a high affinity NH₄⁺ transporter Ta AMT1 or Ta TIP2;1 or the empty vector pYES2 were compared on media with different concentrations of NH₄⁺ at various pH. The ability of Ta TIP2;1 to complement the yeast defective in high affinity NH₄⁺ transport system mep1-3 increased with increasing the pH of the growth medium. The capacity to transport NH₄⁺/NH₃ at an external pH of 7.5 was greater than the capacity of the high affinity transporter (Ta AMT1) from wheat. The data indicate that the de-protonated form NH₃ was transported by Ta TIP2;1. The results are shown in FIG. 4B.

### Example 5

### Cloning into Xenopus Oocytes

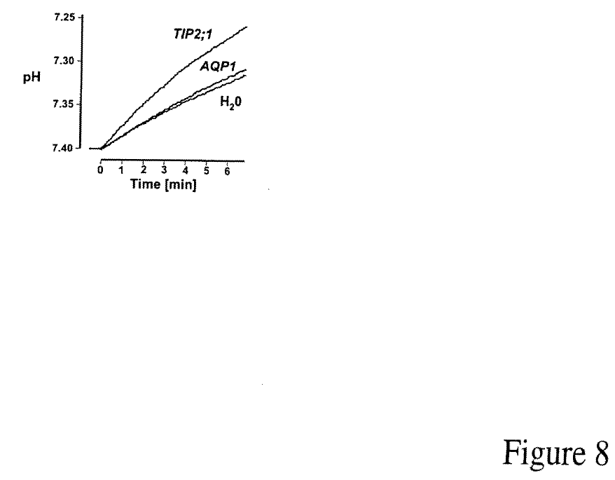

Several different aquaporin cDNAs from various sources were subsequently expressed in Xenopus oocytes and the function of the proteins were investigated. Addition of NH₄⁺ to Xenopus oocytes resulted in a continuous acidification of the medium, in line with the interpretation that NH₃ diffused into the oocyte, leaving H⁺ in the external medium. Acidification was significantly increased after injection with Ta TIP2 mRNA compared to control oocytes injected with water (FIG. 8; Table 1). Expression of human AQP1 did not increase NH₄⁺ induced acidification compared to water injected controls although water transport could be demonstrated for both Ta TIP2;1 and Hs AQP1 mRNA-injected oocytes (Table 1). Expression of Rn AQP3 and Hs AQP9 also resulted in increased medium acidification suggesting that also AQP3 and AQP9 transport NH₄⁺/NH₃.

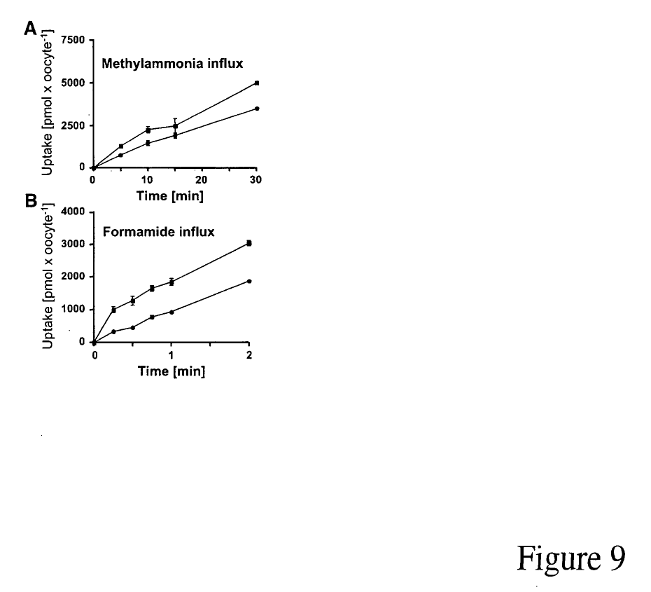

Fluxes of both ¹⁴C-methyl ammonium and ¹⁴C-formamide, two NH₄⁺/NH₃ analogues were measured in oocytes either injected with Ta TIP2;2 mRNA or water as a control. Both, exposure to 20 mM methyl ammonium and 20 mM formamide led to a time dependent accumulation of ¹⁴C in the oocytes. Accumulation by oocytes expressing Ta TIP2;2 was significantly higher than accumulation by control oocytes indicating a specific transport of the two NH₄⁺/NH₃ analogues by Ta TIP2;2. The initial specific uptake of formamide was much higher compared with methyl ammonium. Formamide is a non-charged compound while methyl ammonium in aqueous forms both methyl-NH₃⁺ and methyl-NH₂ with a much higher proportion of the protonated species at neutral pH. Thus the preferred uptake of formamide provides additional evidence that the non-protonated form NH₃ is the substrate transported by Ta TIP2s. The results are shown in FIG. 9.

### Example 6

### Demonstration of Bidirectional Transport of NH₄⁺/NH₃ Through Ta TIP2;1

The yeast Δmep1-3 mutant (31019b) was transformed with either Ta TIP2;1 or an empty vector pYES2 and growth was compared in liquid media with arginine as alternative N-source at various pH. At relatively high pH (7.5), yeast expressing Ta TIP2;1 was strongly delayed in growth in line with the interpretation that NH₄⁺/NH₃ produced in yeast from arginine was secreted into the medium via TIP2;1 resulting in N limitation. The data are illustrated in FIG. 7. Supplementing the media with 2 mM NH₄⁺ completely elevated the growth repression (not shown). The results demonstrate that transport of NH₄⁺/NH₃ through aquaporins is bidirectionally and dependent on both NH₄⁺/NH₃ concentrations and pH differences between the two compartments surrounding the membrane.

### Example 7

### Voltage Clamping of Oocytes Expressing Aquaporin Homologues

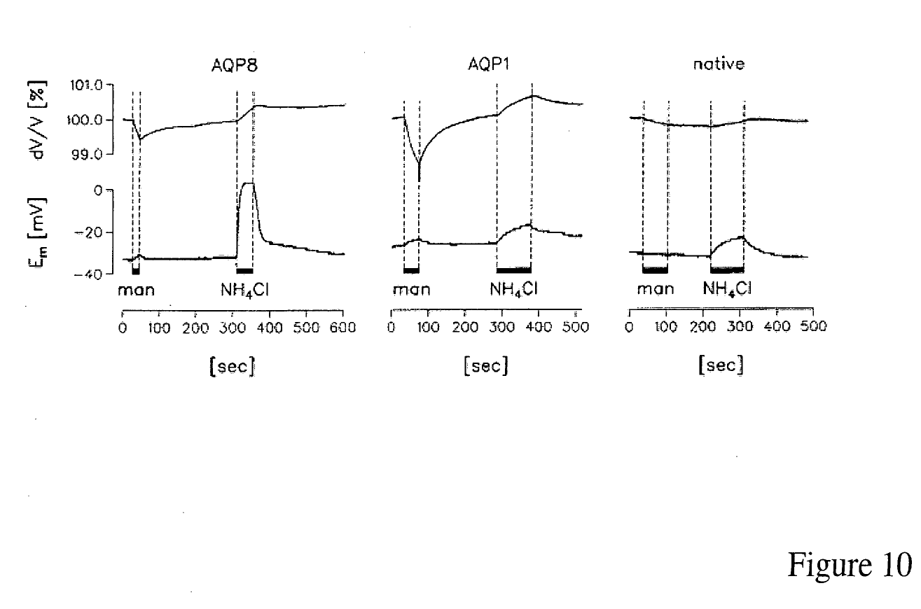

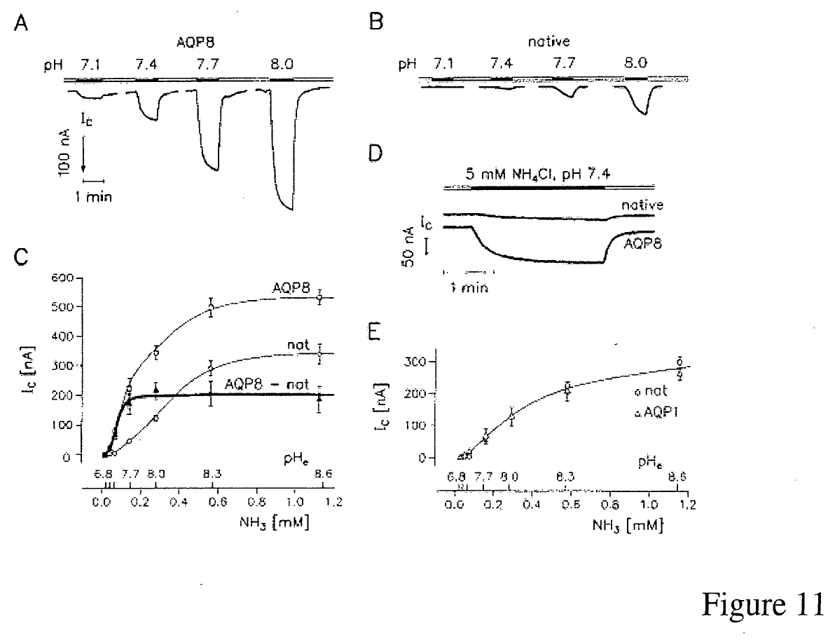

Voltage clamping of oocytes expressing different aquaporins revealed that at increasing concentration addition of NH₄⁺/NH₃ created a positive inward current. The current seemed to be specific for NH₄⁺ since replacing NH₄⁺ by Na⁺ did not lead to the same observation. The data are illustrated in FIGS. 10 and 11.

### Example 8

### Identification of Residues Critical for NH₄⁺/NH₃ Transport Through Aquaporins

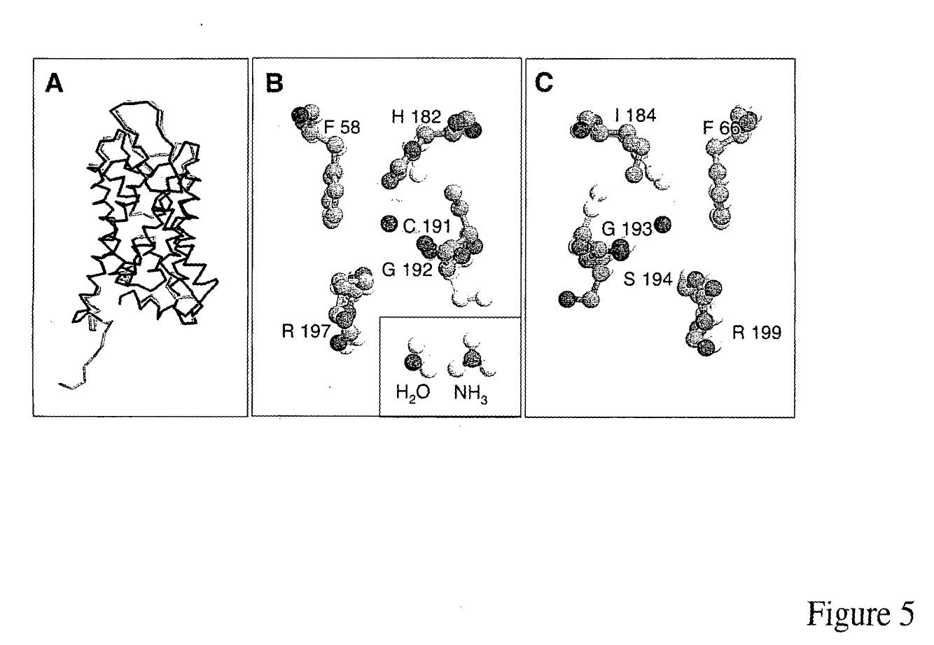

Homology modelling of the sequence of Ta TIP2;1 using the structure of bovine AQP1 lead to the observation, that substitutions on the constriction region of the TIP2 channels result in a wider and more hydrophobic constriction region in TIP2 compared to AQP1. These substitutions were H182 and C191 in AQP1 versus I184 and G193 in TaTIP2s. Results are illustrated in FIG. 5. Strikingly the same substitutions were identified in human AQP8, the isoform of which cDNA complemented the yeast mutant on NH₄⁺ as the sole N source.

Mutating I184 in Ta TIP2;1 into histidine, the corresponding residue of AQP1 significantly decreased NH₄⁺/NH₃ transport when expressed in yeast. Mutating both I184 into histidine and G193 into cysteine resulted in a mutant, which was no longer able to support growth of yeast on NH₄⁺.

### REFERENCES

1. Husted & Schjoerring 1996 Plant Physiol. 112, 67-74.
2. Sener & Malaisse 1980 Diabete Metab 6, 97-101.
3. Sener et al. 1978 J Clin Invest, 868-878.
4. Wall 1997 Am J Physiol 273, 857-868.
5. Watts & Good 1994 J Gen Physiol 103, 917-936.
6. Butterworth 1998 J Inherit Metab Dis 21, 6-20.
7. Zhou & Norenberg 1999 Nuerosci Lett. 276, 145-148.
8. Marini et al. 1997 Mol. Cellular Biol 17, 4282-4293.
9. Michel-Reydellet et al. Mol Gen Genet 258, 671-677.
10. Ninnemann et al. 1994 EMBO J 13, 3464-3471.
11. Sohlenkamp et al. 2000 FEBS Letters 467, 273-278.
12. Ludewig et al. J Biol Chem 277, 13548-13555.
13. Maurel et al. 1993 EMBO J 12, 2241-2247.
14. Loo et al. 2002 J Physiol 542, 53-60.
15. Nakhoul et al. 2001 Am J Physiol Renal Physiol 281, F255-F263.
16. Jauh et al. 1998 PNAS 95, 12995-12999.
17. Jauh et al. 1999 Plant Cell 11, 1867-1882.
18. Wienkoop & Saalbach 2003 Plant Physiol. 131, 1080-1090.
19. Pearson & Lipman, 1988 Methods in Enzymology 183, 63-98.
20. Sambrook et al. 1989 Molecular Cloning: a Laboratory Manual: 2nd edition, Cold Spring Harbor Laboratory Press (or later editions of this work).
21. Asubel et al. eds. 1992 Current Protocols in Molecular Biology, John Wiley & Sons.
22. Morganti et al. 1996, Biotechnology and Applied Biochemistry 23, 67-75.
23. Dunn et al. 1996, Immunotechnology 2, 229-240.
24. Morganti et al. 1998, Biotechnology and Applied Biochemistry 27, 63-70.
25. Guerineau & Mullineaux 1993 In: Plant Molecular Biology Labfax (Croy RRD ed) Oxford, BIOS Scientific Publishers, pp 121-148.
26. Noh & Amasino 1999 Plant Mol. Biol. 41, 181-194.
27. Lindsey & Jones 1989 ‘Plant Biotechnology in Agriculture’ Pub. OU Press, Milton Keynes, UK, p. 120.
28. Caddick et al. 1998, Nature Biotechnology 16, 177-180.
29. Freeman et al. 1984, Plant Cell Physiol. 29, 1353.
30. Kindle 1990, PNAS U.S.A. 87, 1228.
31. Oard 1991, Biotech. Adv. 9, 1-11.
32. Vasil et al. 1984, Cell Culture and Somatic Cell Genetics of Plants, Vol I, II and III, Laboratory Procedures and Their Applications, Academic Press.
33. Weissbach & Weissbach 1989 Methods for Plant Molecular Biology, Academic Press.
34. Shimamoto 1994, Current Opinion in Biotechnology 5, 158-162.
35. Vasil et al. 1992, Bio/Technology 10, 667-674.
36. Vain et al. 1995, Biotechnology Advances 13, 653-671.
37. Vasil 1996, Nature Biotech. 14, 702.
38. Armitage et al. 1992, Nature 357, 80-82.
39. Bourque 1995, Plant Science 105, 125-149.
40. Flavell 1994, PNAS USA 91, 3490-3496.
41. van der Krol et al. 1990 The Plant Cell 2, 291-299.
42. Napoli et al. 1990, The Plant Cell 2, 279-289.
43. Zhang et al. 1992, The Plant Cell 4, 1575-1588.
44. Jaeger 1997 Curr. Opin. Struct. Biol. 7, 324-335
45. Gibson & Shillitoe 1997 Mol. Biotech. 7, 242-251.
46. Quigley et al. 2002 Genome Biol. 3.

TABLE 1

| NH3 | | | | | | |
|---|---|---|---|---|---|---|
| [mM] | pHₑ | AQP8 | AQP9 | AQP3 | TIP2 | AQP1 |
| 0.036 | 6.5 | 0.10 ± 0.06 | 0.08 ± 0.04 | 0.07 ± 0.04 | 0.24 ± 0.05 | −0.01 ± 0.035 |
| | | (5) N.S | (7) N.S. | (6) N.S. | (7) | (6) N.S. |
| | | | | | (p < 0.002) | |
| 0.28 | 7.4 | 1.1 ± 0.48 | 0.9 ± 0.24 | 0.57 ± 0.17 | 1.1 ± 0.24 | 0.24 ± 0.19 |
| | | (6) | (7) | (11) | (7) | (3) N.S. |
| | | (p < 0.03) | (p < 0.005) | (p < 0.005) | (p < 0.00005) | |
| 3.6 | 8.5 | 11.5 ± 3:8 | 7.1 ± 1.5 | 4.7 ± 1.5 | 10.1 ± 2.0 | 3.2 ± 1.8 |
| | | (4) | (7) | (7) | (7) | (5) N.S. |
| | | (p < 0.03) | (p < 0.0005) | (p < 0.02) | (p < 0.002) | |

| | | | | | |
|---|---|---|---|---|---|
| Lₚ | 5.7 ± 0.43 | 1.5 ± 0.17 | 4.1 ± 0.4 | 7.2 ± 0.6 | 5.3 ± 0.5 |
| | (9) | (8) | (14) | (12) | (10) |

1. Use of a polypeptide member of the aquaporin superfamily, or a derivative thereof, as an NH₃/NH₄⁺ transporter,
wherein said polypeptide does not have all of the following amino acid residues at the stated positions using the numbering of bovine AQP1: F58, H182, C191 and R197.

2. Use as claimed in claim 1 wherein the polypeptide does not have both of the following amino acid residues at the stated positions using the numbering of bovine AQP1: H182, C191.

3. Use as claimed in claim 2 wherein the polypeptide has an amino acid residue selected from the following: isoleucine, valine, glycine and alanine, at the stated positions using the numbering of bovine AQP1: 58, 182, 191 and 197.

4. Use as claimed in claim 3 wherein the polypeptide has both of the following amino acid residues at the stated positions using the numbering of bovine AQP1, I182, G191.

5. Use as claimed in claim 1 wherein the polypeptide is a bidirectional NH₃/NH₄⁺ transporter.

6. Use as claimed in claim 5 wherein the transport is driven by a concentration gradient of NH₄⁺, NH₃ and H+ across the membrane.

7. Use as claimed in claim 1 wherein the polypeptide is selected from the TIP(Tonoplast Intrinsic Protein)2 homologues encoded by Ta TIP2;1, Ta TIP2;2 and Ta TIP2;3 deposited in the NCBI database as AY525639, AY525640 and AY535641.

8. Use as claimed in claim 1 wherein the polypeptide is selected from human or animal AQP3, 8 or 9.

9. Use as claimed in claim 1 wherein the polypeptide is a derivative of a polypeptide sequence selected from the list consisting of: the polypeptide shown in FIG. 1; TIP(Tonoplast Intrinsic Protein)₂ homologues encoded by TaTIP2;1, TaTIP2;2 and TaTIP2;3 deposited in the NCBI database as AY525639, AY525640 and AY535641; human or animal AQP3, 8 or 9, which derivative shares at least 60%, 70%, 80%, 90%, 95%, 96%, 97%, 98% or 99% homology with said polypeptide sequence.

10. A process for enhancing the NH₃/NH₄⁺ transport properties of an aquaporin which method comprises modifying the polypeptide or nucleic acid encoding therefore such that said polypeptide does not have all of the following amino acid residues at the stated positions using the numbering of bovine AQP1: F58, H182, C191 and R197.

11. A process as claimed in claim 10 wherein the modification comprises introducing an amino acid residue selected from the following isoleucine, valine, glycine and alanine at one or more of the stated positions using the numbering of bovine AQP1: 58, 182, 191 and 197.

12. A process as claimed in claim 11 wherein the modification comprises introducing the following amino acid residues at the stated positions using the numbering of bovine AQP1, I182, G191.

13. (canceled)

14. (canceled)

15. A method of influencing or affecting NH₃/NH₄⁺ transport across a cell membrane by introducing a heterologous polypeptide as described in claim 1 into the membrane.

16. A method as claimed in claim 15 wherein the polypeptide is provided by expression from a heterologous nucleic acid in the cell.

17. A method as claimed in claim 15 wherein the nucleic acid encodes one or more signal peptides for insertion of the polypeptide into the cell membrane.

18. A method as claimed in claim 15 wherein the cell is in an organism.

19. A method as claimed in claim 18 wherein the organism is plant which is transgenic for the polypeptide.

20. A method as claimed in claim 19 wherein the plant is a transgenic legume which overexpresses the polypeptide.

21. A method as claimed in claim 15 to alleviate a stress or disease condition in a cell characterized by high levels of extracellular or cytoplasmic NH₃/NH₄⁺.

22. A transgenic plant which is transgenic for the heterologous polypeptide as described in claim 1 having modified NH₃/NH₄⁺ transport.

23. A method of treatment of a disorder associated with high levels of NH₄⁺ which method comprises administering a polypeptide as described in claim 1 or nucleic acid encoding therefor.

24. A method as claimed in claim 23 wherein the disorder is to metabolic acidosis in the kidney, central nervous system dysfunction, Alzheimer's Type II astrocytosis, and brain oedema.

25. A polypeptide as described in claim 1 for screening for a compound capable of influencing or affecting the NH₃/NH₄⁺ transport in a cell or organism.

26. (canceled)

27. A method of identifying and/or cloning, from a eukaryotic cell, a nucleic acid molecule encoding a NH₃/NH₄⁺ transporter, which method employs a nucleic acid molecule encoding a polypeptide as described in claim 1.

28. Use of a nucleotide sequences complementary to a sequence encoding a polypeptide as described in claim 1 for down regulation of NH₃/NH₄⁺ transport in a cell in which said complementary nucleotide sequence is introduced.


SEQUENCE LISTING (25 sequences)

SEQ ID NO: 1 - protein, 269 aa, Homo sapiens
MASEFKKKLFWRAVVAEFLATTLFVFISIGSALGFKYPVGNNQTAVQDNVKVSLAFGLSIATLAQSVGHISGAHLNPAVTLGLLLSCQISIFRALMYIIAQCVGAIVATAILSGITSSLTGNSLGRNDLADGVNSGQGLGIEIIGTLQLVLCVLATTDRRRRDLGGSAPLAIGLSVALGHLLAIDYTGCGINPARSFGSAVITHNFSNHWIFWVGPFIGGALAVLIYDFILAPRSSDLTDRVKVWTSGQVEEYDLDADDINSRVEMKPK

SEQ ID NO: 2 - protein, 271 aa, Homo sapiens
MWELRSIAFSRAVFAEFLATLLFVFFGLGSALNWPQALPSVLQIAMAFGLGIGTLVQALGHISGAHINPAVTVACLVGCHVSVLRAAFYVAAQLLGAVAGAALLHEITPADIRGDLAVNALSNSTTAGQAVTVELFLTLQLVLCIFASTDERRGENPGTPALSIGFSVALGHLLGIHYTGCSMNPARSLAPAVVTGKFDDHWVFWIGPLVGAILGSLLYNYVLFPPAKSLSERLAVLKGLEPDTDWEEREVRRRQSVELHSPQSLPRGTKA

SEQ ID NO: 3 - protein, 265 aa, Homo sapiens
MKKEVCSVAFLKAVFAEFLATLIFVFFGLGSALKWPSALPTILQIALAFGLAIGTLAQALGPVSGGHINPAITLALLVGNQISLLRAFFYVAAQLVGAIAGAGILYGVAPLNARGNLAVNALNNNTTQGQAMVVELILTFQLALCIFASTDSRRTSPVGSPALSIGLSVTLGHLVGIYFTGCSMNPARSFGPAVVMNRFSPAHWVFWVGPIVGAVLAAILYFYLLFPNSLSLSERVAIIKGTYEPDEDWEEQREERKKTMELTTR

SEQ ID NO: 4 - protein, 261 aa, Homo sapiens
MSGEIAMCEPEFGNDKAREPSVGGRWRVSWYERFVQPCLVELLGSALFIFIGCLSVIENGTDTGLLQPALAHGLALGLVIATLGNISGGHFNPAVSLAAMLIGGLNLVMLLPYWVSQLLGGMLGAALAKAVSPEERFWNASGAAFVTVQEQGQVAGALVAEIILTTLLALAVCMGAINEKTKGPLAPFSIGFAVTVDILAGGPVSGGCMNPARAFGPAVVANHWNFHWIYWLGPLLAGLLVGLLIRCFIGDGKTRLILKAR

SEQ ID NO: 5 - protein, 295 aa, Homo sapiens
MQPEGAEKGKSFKQRLVLKSSLAKETLSEFLGTFILIVLGCGCVAQAILSRGRFGGVITINVGFSMAVAMAIYVAGGVSGGHINPAVSLAMCLFGRMKWFKLPFYVGAQFLGAFVGAATVFGIYYDGLMSFAGGKLLIVGENATAHIFATYPAPYLSLANAFADQVVATMILLIIVFAIFDSRNLGAPRGLEPIAIGLLIIVIASSLGLNSGCAMNPARDLSPRLFTALAGWGFEVFRAGNNFWWIPVVGPLVGAVIGGLIYVLVIEIHHPEPDSVFKAEQSEDKPEKYELSVIM

SEQ ID NO: 6 - protein, 292 aa, Homo sapiens
MGRQKELVSRCGEMLHIRYRLLRQALAECLGTLILVMFGCGSVAQVVLSRGTHGGFLTINLAFGFAVTLGILIAGQVSGAHLNPAVTFAMCFLAREPWIKLPIYTLAQTLGAFLGAGIVFGLYYDAIWHFADNQLFVSGPNGTAGIFATYPSGHLDMINGFFDQFIGTASLIVCVLAIVDPYNNPVPRGLEAFTVGLVVLVIGTSMGFNSGYAVNPARDFGPRLFTALAGWGSAVFTTGQHWWWVPIVSPLLGSIAGVFVYQLMIGCHLEQPPPSNEEENVKLAHVKHKEQI

SEQ ID NO: 7 - protein, 323 aa, Homo sapiens
MSDRPTARRWGKCGPLCTRENIMVAFKGVWTQAFWKAVTAEFLAMLIFVLLSLGSTINWGGTEKPLPVDMVLISLCFGLSIATMVQCFGHISGGHINPAVTVAMVCTRKISIAKSVFYIAAQCLGAIIGAGILYLVTPPSVVGGLGVTMVHGNLTAGHGLLVELIITFQLVFTIFASCDSKRTDVTGSIALAIGFSVAIGHLFAINYTGASMNPARSFGPAVIMGNWENHWIYWVGPIIGAVLAGGLYEYVFCPDVEFKRRFKEAFSKAAQQTKGSYMEVEDNRSQVETDDLILKPGVVHVIDVDRGEEKKGKDQSGEVLSSV

SEQ ID NO: 8 - protein, 271 aa, Bos taurus
MASEFKKKLFWRAVVAEFLAMILFIFISIGSALGFHYPIKSNQTTGAVQDNVKVSLAFGLSIATLAQSVGHISGAHLNPAVTLGLLLSCQISVLRAIMYIIAQCVGAIVATAILSGITSSLPDNSLGLNALAPGVNSGQGLGIEIIGTLQLVLCVLATTDRRRRDLGGSGPLAIGFSVALGHLLAIDYTGCGINPARSFGSSVITHNFQDHWIFWVGPFIGAALAVLIYDFILAPRSSDLTDRVKVWTSGQVEEYDLDADDINSRVEMKPK

SEQ ID NO: 9 - protein, 248 aa, Triticum aestivum
MPGSIAFGRFDDSFSLASFRAYIAEFISTLIFVFAGVGSVIAYTKVSGGAPLDPSGLIAVAICHGFGLFVAVAIGANISGGHVNPAVTFGLALGGQITILTGIFYWVAQLLGAIVGAFLVQFCTGVATPTHGLSGVGAFEGVVMEIIVTFGLVYTVYATAADPKKGSLGTIAPIAIGFIVGANILVAGPFSGGSMNPARSFGPAVASGDFTNIWVYWAGPLIGGGLAGVVYRYLYMWDDHSPVAGNDY

SEQ ID NO: 10 - protein, 281 aa, Escherichia coli
MSQTSTLKGQCIAEFLGTGLLIFFGVGCVAALKVAGASFGQWEISVIWGLGVAMAIYLTAGVSGAHLNPAVTIALWLFACFDKRKVIPFIVSQVAGAFCAAALVYGLYYNLFFDFEQTHHIVRGSVESVDLAGTFSTYPNPHINFVQAFAVEMVITAILMGLILALTDDGNGVPRGPLAPLLIGLLIAVIGASMGPLTGFAMNPARDFGPKVFAWLAGWGNVAFTGGRDIPYFLVPLFGPIVGAIVGAFAYRKLIGRHLPCDICVVEEKETTTPSEQKASL

SEQ ID NO: 11 - protein, 289 aa, Saccharomyces cerevisiae
MSNESNDLEKNISHLDPTGVDNAYIPPEQPETKHSRFNIDRDTLRNHFIAAVGEFCGTFMFLWCAYVICNVANHDVALTTEPEGSHPGQLIMIALGFGFSVMFSIWCFAGVSGGALNPAVSLSLCLARAISPARCVVMWFPQIIAGMAAGGAASAMTPGKVLFTNALGLGCSRSRGLFLEMFGTAVLCLTVLMTAVEKRETNFMAALPIGISLFMAHMALTGYTGTGVNPARSLGAAVAARYFPHYHWIYWIGPLLGAFLAWSVWQLLQILDYTTYVNAEKAAGQKKED

SEQ ID NO: 12 - protein, 286 aa, Arabidopsis thaliana
MEGKEEDVRVGANKFPERQPIGTSAQSDKDYKEPPPAPLFEPGELASWSFWRAGIAEFIATFLFLYITVLTVMGVKRSPNMCASVGIQGIAWAFGGMIFALVYCTAGISGGHINPAVTFGLFLARKLSLTRAVYYIVMQCLGAICGAGVVKGFQPKQYQALGGGANTIAHGYTKGSGLGAEIIGTFVLVYTVFSATDAKRNARDSHVPILAPLPIGFAVFLVHLATIPITGTGINPARSLGAAIIFNKDNAWDDHWVFWVGPFIGAALAALYHVIVIRAIPFKSRS

SEQ ID NO: 13 - protein, 287 aa, Arabidopsis thaliana
MAKDVEAVPGEGFQTRDYQDPPPAPFIDGAELKKWSFYRAVIAEFVATLLFLYITVLTVIGYKIQSDTDAGGVDCGGVGILGIAWAFGGMIFILVYCTAGISGGHINPAVTFGLFLARKVSLPRALLYIIAQCLGAICGVGFVKAFQSSYYTRYGGGANSLADGYSTGTGLAAEIIGTFVLVYTVFSATDPKRSARDSHVPVLAPLPIGFAVFMVHLATIPITGTGINPARSFGAAVIYNKSKPWDDHWIFWVGPFIGAAIAAFYHQFVLRASGSKSLGSFRSAANV

SEQ ID NO: 14 - protein, 250 aa, Arabidopsis thaliana
MAGVAFGSFDDSFSLASLRAYLAEFISTLLFVFAGVGSAIAYAKLTSDAALDTPGLVAIAVCHGFALFVAVAIGANISGGHVNPAVTFGLAVGGQITVITGVFYWIAQLLGSTAACFLLKYVTGGLAVPTHSVAAGLGSIEGVVMEIIITFALVYTVYATAADPKKGSLGTIAPLAIGLIVGANILAAGPFSGGSMNPARSFGPAVAAGDFSGHWVYWVGPLIGGGLAGLIYGNVFMGSSEHVPLASADF

SEQ ID NO: 15 - protein, 380 aa, Artificial sequence
  Majority or Consensus Sequence
MAGXXXXXXXXXXXXXXXXXXXXXXXXXXXFKDXXXXAXXXXXXXXXXXFWRAVLAEFLATLLFVFIGVGSVIGXXXXXXAXXXLALXXPGGALAIALAFGLSIATLIQATGGISGGHINPAVTLALLLGGQISLLRAVFYIVAQLLGAIAGAGLVKGVXXXXXXXXTPGLAXXVGXGLGXNALXXXXXXXAPGLTAGQGLVVEIIGTFQLVLTVFATTDDKRRGXXXSLGGLAPLAIGFSVALGHLAAIPYTGCGMNPARSFGPAVITGNFAXXXXXXXXXXDHWIFWVGPLIGAALAALLYGFVLRXXXXXXXXXXXXXXXXXXXXXXXXXXXXXXXXXXXXXXXXXXXXXXXXXXXXXXXXXXXXXXXXXXXXXXXX

SEQ ID NO: 16 - protein, 22 aa, Triticum aestivum
GFIVGANILVAGPFSGGSMNPA

SEQ ID NO: 17 - protein, 22 aa, Arabidopsis thaliana
GFAVFLVHLATIPITGTGINPA

SEQ ID NO: 18 - protein, 22 aa, Arabidopsis thaliana
GFAVFMVHLATIPITGTGINPA

SEQ ID NO: 19 - protein, 22 aa, Homo sapiens
GLSVALGHLLAIDYTGCGINPA

SEQ ID NO: 20 - protein, 22 aa, Homo sapiens
GFSVALGHLLGIHYTGCSMNPA

SEQ ID NO: 21 - protein, 22 aa, Rattus norvegicus
GLVVLVIGTSMGFNSGYAVNPA

SEQ ID NO: 22 - protein, 22 aa, Rattus norvegicus
GFSVAIGHLFAINYTGASMNPA

SEQ ID NO: 23 - protein, 22 aa, Homo sapiens
GLSVTLGHLVGIYFTGCSMNPA

SEQ ID NO: 24 - protein, 22 aa, Homo sapiens
GFAVTVDILAGGPVSGGCMNPA

SEQ ID NO: 25 - protein, 22 aa, Homo sapiens
GLLIIVIASSLGLNSGCAMNPA